(19) United States
(12) Patent Application Publication
(10) Pub. No.: US 2025/0255733 A1
(43) Pub. Date: Aug. 14, 2025

(54) BONE MILL APPARATUS

(71) Applicant: Warsaw Orthopedic, Inc., Warsaw, IN (US)

(72) Inventors: David R. Kaes, Toms River, NJ (US); Wei Cai, New Hope, PA (US); Rudolf H. Duke, Brick Township, NJ (US); Luke H. Parsons, Brick Township, NJ (US)

(21) Appl. No.: 19/053,204

(22) Filed: Feb. 13, 2025

Related U.S. Application Data
(60) Provisional application No. 63/552,715, filed on Feb. 13, 2024.

(51) Int. Cl. A61F 2/46

(52) U.S. Cl. CPC A61F 2/4644; A61F 2002/4645

(57) ABSTRACT

Present embodiments are directed to facilitating and improving bone mill functionality. For example, present embodiments provide for improving yields in bone fibers produced from a donor sample specimen, while additionally reducing the operating duration used to process the sample. In part, present embodiments provide these improvements by providing for hands-free operation of the bone mill, and providing for interchangeability and availability of heavy-wear components. In some embodiments, the process may provide a consistent force biasing a bone sample against a cutter while reducing opportunities for operator accidents.

### CROSS-REFERENCE TO RELATED APPLICATION

This application claims priority and benefit of U.S. Provisional Patent Application No. 63/552,715, entitled “BONE MILL APPARATUS,” filed on Feb. 13, 2024, which is incorporated herein by reference in its entirety.

### TECHNICAL FIELD

This invention relates to an apparatus for cutting bone, and more particularly, to an apparatus for milling bone fragments which may subsequently be demineralized and used to produce shaped materials or used in other contexts where bone matters or fibers are employed.

### BACKGROUND

This section is intended to introduce the reader to various aspects of art that may be related to various aspects of the present techniques, which are described and/or claimed below. This discussion is believed to be helpful in providing the reader with background information to facilitate a better understanding of the various aspects of the present techniques. Accordingly, it should be understood that these statements are to be read in this light, and not as admission of prior art.

The medical industry has various uses for bone fragments, which may be produced by milling or grinding larger pieces of bone to form such fragments. By way of example, bone tissue collected from donor material during a milling or grinding process may help medical personnel treat patients with a variety of skeletal defects, diseases, or injuries. While operating traditionally, a bone mill produces valuable bone fibers that form the foundation of bone grafts that help a recipient re-grow bone material or otherwise receive treatment for a skeletal ailment. However, traditional bone mills produce yields that are variable and irregular, and often lead to generation of unwanted fiber materials that have to be scrapped. Additionally, operating a traditional bone mill is a difficult, typically manual, process that may lead to operator injuries. Accordingly, it is now recognized that new solutions are needed to improve this area of the medical industry.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

Present embodiments are directed to facilitating and improving bone mill functionality. For example, present embodiments provide for improving yields in bone fibers produced from a donor sample specimen, while additionally reducing the operating duration used to process the sample. In part, present embodiments provide these improvements by providing for hands-free operation of the bone mill, and providing for interchangcability and availability of heavy-wear components. In some embodiments, the process may provide a consistent force biasing a bone sample against a cutter while reducing opportunities for operator accidents.

In a non-limiting embodiment, a bone milling apparatus includes a support housing such that the housing includes a base portion with a top surface, a top portion vertically spaced from the base portion, and support braces configured to couple the top portion with the base portion, and a cutter configured to rotate about a rotational axis. Additionally, the bone milling apparatus includes a push block assembly disposed on top of the base portion, such that the push block assembly is configured to translate along the top surface of the base portion, thereby enacting a force against a bone segment during use to keep the bone segment in contact with the cutter, a push block motor communicatively coupled to the push block assembly, and a drive motor communicatively coupled to the cutter, such that the drive motor is configured to rotate the cutter about the rotational axis.

In a non-limiting embodiment, a method includes placing a bone segment into an input chute of a bone milling apparatus, such that the input chute is configured to direct the bone segment to be positioned against a push block of a push block assembly, powering on a drive motor that is coupled to a cutter shaft, such that the drive motor is configured to output a rotational force to the cutter shaft, and outputting the rotational force to the cutter shaft, such that the cutter shaft begins to rotate at a specified rotational speed and a specified torque. Additionally, the method includes powering one a push block motor that is coupled to the push block assembly, such that the push block motor is configured to directly or indirectly output an axial force to the push block assembly, outputting the axial force to the push block assembly, such that the axial force keeps the bone segment in contact with a cutter disposed on the cutter shaft, and milling the bone segment and generating bone fibers as a result of the push block assembly applying the axial force to the bone segment against the cutter.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is an exploded view of a bone milling apparatus, in accordance with aspects of the present techniques;

FIG. 2 is a schematic view of an embodiment of a processor-based control system for an automated bone milling apparatus, in accordance with aspects of the present techniques;

FIG. 3 is a block diagram of example components that may be used in the control system of FIG. 2, in accordance with aspects of the present techniques;

FIG. 4 is a flowchart of an embodiment of a feedback-based method for controlling a bone billing apparatus used to harvest bone tissue fibers from a sample specimen, in accordance with aspects of the present techniques;

FIG. 5 is a front view of an embodiment of the bone milling apparatus in various stages of operation, in accordance with aspects of the present techniques; and

FIG. 6 is a section view of an embodiment of a bearing housing assembly utilized in an embodiment of the bone milling apparatus, in accordance with aspects of the present techniques.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

Present embodiments are directed to facilitating and improving bone mill functionality. For example, present embodiments provide for improving yields in bone fibers produced from a donor sample specimen, while additionally reducing the operating duration used to process the sample. In part, present embodiments provide these improvements by implementing an automated process of the bone mill operation that utilizes force feedback control. Further, the force feedback control can adapt and compensate for changes during the milling process, including but not limited to automatically adjusting a feed rate or a spindle speed of the mill in response to readings from a force sensor. By utilizing aspects of the present techniques, yields of bone fiber that can be produced from donor tissue may increase, while reducing the operating duration.

Also, present embodiments provide for hands-free operation of the bone mill, introducing automatic actuation of the cutting mechanism and/or the feed portion of the mill. In part, the hands-free operation is achieved through the previously discussed implementation of force feedback control. While prior approaches to operating a bone mill required an operator to mechanically actuate a crank to spin a cutter or advance a push block to position a piece of donor tissue against the cutting mechanism, present embodiments eliminate this requirement, and as a result, reduces opportunities for accidents or injuries. Further, the automatic actuation enables the bone mill to control various components during operation to provide a consistent force biasing a bone sample against a cutter.

Additionally or alternatively, present embodiments provide for improvements to the interchangeability and availability of heavy wear and expendable items. For example, the bone mill is designed to accept cutting mechanisms that are readily available, while still maintaining the capability to accept a custom-manufactured cutting mechanism. In one embodiment, the cutting mechanism is configured in a manner such that the blades are oriented in a helical geometry, enabling the cutting mechanism to cut fibers from the donor tissue while avoiding premature breakage or snapping of the bone fibers. Further, the cutting mechanism can be indexed and positioned in a plurality of angles and configurations to accommodate for varying parameters for fiber length, geometry, thickness, and other suitable output variables.

Also, certain embodiments may be configured to operate in a cleanroom environment, and adhere to guidelines in place that govern ISO 5/Class 100 work environments. For example, the bone mill may be configured to extend through a dividing wall such that a portion of the bone mill that includes cutting mechanism and bone specimen extends into the cleanroom environment, and another portion that includes actuators and working parts of the bone mill may operate outside the cleanroom environment. Further, individual components of the bone mill are configured to be removed and cleaned separately from the bone mill and each component may be sterilized individually.

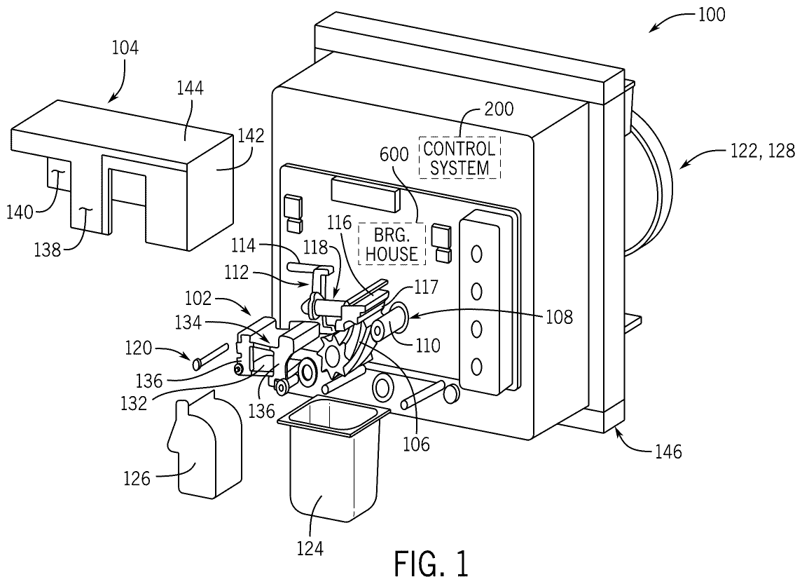

Turning to the drawings, FIG. 1 is an exploded view of the bone milling apparatus 100 according to an embodiment of the present technique. The bone milling apparatus may include a housing 102, a cover 104, a cutter 106, a push block assembly 110, a drive motor 122, a push block motor 128, a control system 200, a bearing support assembly 148 and a fiber pan 124. The housing 102 may be configured to provide a support structure for the cutter 106 and the push block assembly 110. In certain embodiments, the housing 102 includes a base portion 132 disposed in a horizontal orientation, and a top portion 134 similarly disposed in a horizontal orientation, vertically offset above the base portion 132 by a specified distance. In other embodiments, the base portion 132 and the top portion 134 may be parallel with respect to each other, and the specified distance may create a cavity suitable to at least partially house the push block assembly 110, the cutter 106, or both. The housing 102 may also include a plurality of support braces 136 configured to fix the top portion 134 in a position sufficiently apart from the base portion 132 such that the aforementioned cavity may be created. Additionally or alternatively, the support braces 136 may be configured to provide rigidity to the housing 102 and positioned to counteract any reaction forces experienced by the housing 102 during the bone milling operation. The housing 102, base portion, 132, top portion, 134, and support braces 136 may be made from any suitable material, such as but not limited to metal, metal alloy, ceramics, carbon fiber compositions, polymeric compositions (e.g., plastic), wood, or any other suitable material or combinations thereof.

In certain embodiments, the bone milling apparatus 100 may include a cover 104. The cover 104 is configured to be positioned around the housing 102, cutter 106, and push block assembly 110 such that the internal moving components are protected from any elements in a cutting room environment. Additionally or alternatively, the cover 104 is configured to act as a barrier during milling operation to prevent shavings from a donor sample or other by-products of the milling process to enter the environment. In a non-limiting embodiment, the cover 104 includes a front panel 138, a back panel 140, at least one side panel 142, and a top panel 144. In other embodiments, the front panel is configured to have a plurality of cutouts positioned appropriately such that an operator may add or remove a bone segment, or otherwise access input areas of the bone milling apparatus. The cover 104 and its associated panels 138, 140, 142, 144 may be made from any suitable material, such as but not limited to metal, metal alloy, ceramics, carbon fiber compositions, polymeric compositions (e.g., plastic), wood, or any suitable material or combinations thereof. The cover 104 may be configured to removably couple to the bone milling apparatus, enabling removal when maintenance or other procedures require more comprehensive access to the internal components.

In a non-limiting embodiment, the bone milling apparatus includes a cutter 106 disposed on a cutter shaft 108. The cutter 106 may be coupled with or integrally attached to the cutter shaft 108, such that the cutter rotates with, and is rotatable along the axis of, the cutter shaft at a desired rotational speed. The cutter shaft 108 may protrude from an aperture in a barrier wall 146 such that the cutter shaft extends a specified distance into the cutting room environment, enabling the cutter 106 to rotate and apply a cutting force to a bone segment. The cutter shaft 108 may be configured to accept cutters 106 that are readily available as a standard option (i.e. standard size in a catalog, available online as a default size, etc.) or may accept cutters 106 that include customized specifications that are detailed for a specific embodiment. In some embodiments, the cutter 106 may be decoupled from the cutter shaft 108 so that the cutter may be cleaned separately. The cutter 106 may be made from any suitable material, such as but not limited to metal, metal alloy, or any combination thereof.

The cutter 106 may be any suitable length and diameter. In one non-limiting embodiment, the cutter 106 may be a length that is equal to or greater than the length of the bone segments for which the bone milling apparatus 100 is designed to receive for milling. For example, in one embodiment, the cutter 106 may have a length, along its axis of rotation, of between three and one half (3½) to four (4) inches. Additionally or alternatively, the cutter 106 may have any suitable diameter. In a non-limiting embodiment, the cutter may have a diameter of between two and one half (2½) to three (3) inches. However, any suitable length or diameter of cutter 106 is considered within the scope of the various embodiments of the present techniques.

Further, the cutter 106 may have any suitable number of teeth or bladed edges. In some embodiments, the cutter 106 may have between two (2) and ten (10) teeth or bladed edges. However, a single tooth or bladed edge as well as greater than ten (10) teeth or bladed edges are considered within the scope of the various embodiments of the present techniques. In a non-limiting embodiment, the cutter includes eight (8) teeth or bladed edges. Also, the teeth or bladed edges may be configured in a suitable arrangement along the axis of rotation. In certain embodiments, the teeth or bladed edges of the cutter 106 may be configured in a helical pattern around the cutter 106. In certain embodiments, the helical pattern of the teeth or bladed edges may traverse the length of the cutter 106 at a helix angle of thirty degrees (30°). However, helix angles above and below thirty degrees (30°) are considered within the scope of the various embodiments of the present techniques. The teeth or bladed edges of the cutter 106 may be configured depending on a desired specification of the fibers that result from the milling process. For example, the helix angle of the teeth or bladed edges may be one factor in determining a thickness of the fibers for a given cutter rotational speed.

In certain embodiments, the cutter shaft may be connected to a drive motor 122, such as a variable speed drive motor. In other embodiments, the cutter shaft may be configured to be actuated manually with a crank, or other suitable mechanism. The drive motor 122 may be operated manually, electrically, or by a computing device. In a non-limiting embodiment, the drive motor 122 may be isolated from the cutting room environment such as in an isolation chamber or adjacent room, so that any contaminants (e.g. dust, grease, shavings, or fumes) created by the drive motor 122 may be kept away from the cutting room environment, which may be a clean room or sterile environment.

The bone milling apparatus 100 includes a push block assembly 110 configured to hold or push the bone segment against the cutter 106 during operation of the bone mill. The push block assembly includes a crank arm 112, a crank handle 114, a push block 116, and a drive screw 118. In certain embodiments, the crank arm 112 is coupled to the crank handle 114 in a perpendicular orientation and in a position such that the crank handle 114 is positioned at an end of the crank arm 112. In other embodiments, the crank handle 114 is positioned in other suitable positions along the length of the crank arm 112, as long as a force applied along the length of the crank handle 114 transfers a tangential force causing the crank arm 112 to rotate around an end opposite to the end where the crank handle 114 mounts.

As a result of the application of the tangential force, the crank arm 112 introduces a rotational motion to the drive screw 118. At a first end, the drive screw 118 is removably coupled to the end of the crank arm 112 opposite to the crank handle 114, and at a second end, the drive screw 118 is removably coupled to the push block 116. In certain embodiments, the drive screw 118 is configured to transfer rotational motion into translational motion, or an axial force. For example, as a result of the crank arm 112 introducing a rotational motion to the first end of the drive screw 118, the second end of the drive screw 118 translates in an axial direction in relation to the crank handle, which in turn transfers an axial force to the push block 116. In other embodiments, the drive screw may be dimensioned in such a manner such that the geometry of the screw may sufficiently counteract any forces that may propagate from the cutter 106 through the push block 116.

The push block assembly 110 includes the push block 116 that may be configured to engage the bone segment and hold the sample against the cutter 106. Additionally or alternatively, the push block 116 includes an interface surface 117 configured to include one or more engaging features that assist in maintaining the sample against the cutter 106. In certain embodiments, the engaging features include serrations, spikes, nodules, one or more textured surfaces, knurling, or the like, or any combinations thereof. The engaging features of the interface surface 117 may be integral to the push block 116, or may removably attach to the push block. The engaging features of the interface surface may be made of similar or different material than the push block 116. Further, the geometry of the push block 116 may be configured such that the profile of the push block matches or is complementary to the diameter of the cutter 106. For example, the profile geometry of the interface surface 117 may be defined in part by a radius characteristic or parameter that matches or approximates (i.e., matches within a tolerance such as a manufacturing tolerance) the diameter of the cutter 106, such that when the drive screw 118 extends the push block 116 to a maximum length, the interface surface 117 may closely fit around a portion of the cutter 106, enabling the bone segment to be nearly completely be milled with little or no residual bone material being unprocessed.

In certain embodiments, the push block assembly 110 may be connected to a push block motor 128, such as a variable speed drive motor. As described above, similar to the drive motor 122, the push block motor 128 may be operated manually, electrically, or by an electromechanical controller or computing device. In a non-limiting embodiment, the push block motor 128 may be isolated from the cutting room environment such as in an isolation chamber or adjacent room, so that any contaminants (e.g. dust, grease, shavings, fumes, etc.) created by the push block motor 128 may be kept away from the cutting room environment.

The bone milling apparatus 100 may include a feed chute 126. Generally, the feed chute 126 may be include an opening, bore, or aperture positioned proximate to the housing 102. In a non-limiting embodiment, the feed chute 126 may be configured to receive a bone segment having a length corresponding approximately to the length of the cutter 106 (or shorter), and to present the sample in an orientation such that the length of the sample is substantially parallel to the rotational axis of the cutter 106 during operation. In certain embodiments, the bone segment is a portion of a bone, and the results from the milling operation produces fibers of a specified length and thickness. As the fibers are produced, they are collected in a fiber pan 124 disposed in a suitable position proximate to the cutter 106 and bone segment such that the fiber pan catches the fibers produced from the operation. After the fibers from the sample are produced, they are transferred in the fiber pan 124 to a processing area so that they may be utilized, such as in the production of shaped osteogenic materials.

In the illustrated embodiment, a bearing support assembly 148 is included in the bone milling apparatus 100. As will be discussed in further detail below, the bearing support housing 14 includes a plurality of bearings configured to provide support to the cutter shaft 108 during operation of the bone mill. Also, a control system 200 is included in the bone milling apparatus 100. As will be discussed in further detail below, the control system 200 includes a controller, which in some embodiments includes a processor and a memory configured to provide instructions to various components of the bone milling apparatus 100. In other embodiments, the control system may utilize a specialized integrated circuit (e.g., an application specific integrated circuit (ASIC)) designed to generate a control signal in response to a feedback signal as opposed to or in addition to a processor. In certain embodiments, the control system 200 may output instructions to the drive motor 122 and/or the push block motor 128 that controls the electrical power (i.e., a current, a voltage) transmitted to the actuating components of the bone milling apparatus 100. The control system 200 may include a plurality of controllers, such that a controller is configured to output instructions to an individual actuator. In other embodiments, a single controller is configured to output instructions to the various actuating components of the bone milling apparatus.

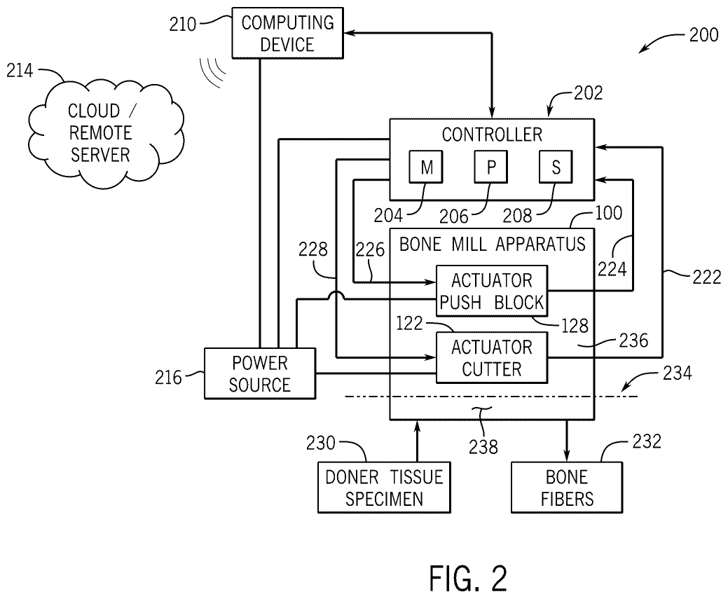

FIG. 2 is a schematic view of the elements of the control system 200 in accordance with aspects of the present techniques. In the illustrated embodiment, the control system 200 for the bone milling apparatus 100 is depicted. The control system 200 includes a controller 202 that is communicatively or electrically coupled to the drive motor 122 that actuates the cutter 106, and communicatively or electrically coupled to the push block motor 128 that actuates the push block assembly 110. As discussed in more detail below, the controller 202 may be configured to output instructions to the motors in the form of control signals along control paths 226, 228, and may be configured to receive feedback along communication paths 222, 224 that are indicative of operating conditions while the bone milling apparatus 100 is in operation. The controller 202 includes a processor 206, a memory 204, and a sensor 208 that are configured to work together to provide instructions to the various components of the bone milling apparatus 100. For example, the controller 202 may be programmed (e.g., via computer readable code or instructions stored on the memory 204, such as a non-transitory computer readable medium, and executable by the processor 206 and/or by circuit design of an ASIC) to provide signals for controlling the motors 122, 128. In certain embodiments, the controller 202 may be programmed according to a specific configuration desired for a particular application. Additionally or alternatively, the sensor 208 may be configured to detect external inputs, reference signals, or other suitable signals that are indicative of the performance of the bone milling apparatus 100. In a non-limiting embodiment, the controller 202 may be configured to respond to external inputs and reference signals detected by the sensor 208, and the controller may be configured to respond in a programmed or pre-determined manner according to a set of operating parameters. For example, the operating parameters may determine the speed or torque of the motors 122, 128 or may determine how the controller 202 responds to various external inputs detected by the sensor 208. With feedback data from the sensor 208, the control system 200 may keep detailed track of the various conditions under which the bone milling apparatus 100 is operating. For example, the feedback data may include conditions such as actual motor speed, voltage, frequency, power quality, alarm conditions, etc. In some embodiments, the feedback data may be communicated back to one or more computing devices 210 for additional analysis.

The computing device 210 may be communicatively coupled to the controller 202 via a wired or wireless connection. The computing device 210 may receive inputs from a user defining a bone milling operation using a native application running on the computing device 210 or using a website accessible via a browser application, a software application, or the like. The user may define a series of steps and processes that define the bone milling operation by writing code, interacting with a visual programming interface, inputting or selecting values via a graphical user interface, or providing some other inputs. The user may use software and/or software services to create, analyze, and otherwise develop the operation. The computing device 210 may send a set of operating parameters to the controller 202 for execution. Execution of the operation causes the controller 202 to control the actuating components (i.e. the drive motor 122 and the push block motor 128) through performance of one or more tasks and/or processes. The controller 202, via the sensor 208, may collect feedback data during execution of the operation, and the feedback data may be provided back to the computing device 210 for analysis. In certain embodiments, the feedback data may include one or more execution times, one or more alerts, one or more error messages, one or more alarm conditions, one or more motor speeds, one or more voltages, one or more frequencies, and so forth.

The computing device 210 may be communicatively coupled to a cloud server 214 or remote server via the internet, or some other network. In one embodiment, the cloud server 214 may be operated by the manufacturer of the controller 202, a software provider, a seller of the controller 202, a service provider, an operator of the controller 202, owner of the controller 202, etc. The cloud server may be used to help users create and modify different operating programs or control feedback “recipes” to be utilized by the bone milling apparatus 100, to help troubleshoot any problems that arise with the controller, develop policies, or to provide other services. The remote/cloud server 214 may be one or more servers operated by the manufacturer, software provider, seller, service provider, operator, or owner of the controller 202. The remote/cloud server 214 may be disposed at a facility owned and/or operated by the manufacturer, software provider, seller, service provider, operator, or owner of the controller 202. In other embodiments, the remote/cloud server 214 may be disposed in a datacenter in which the manufacturer, software provider, seller, service provider, operator, or owner of the controller 202 owns or rents server space. In further embodiments, the remote/cloud server 214 may include multiple servers operating in one or more data center to provide a cloud computing environment.

The control system 200 includes a power source 216, which is configured to provide electrical power to various components of the control system 200. The power source 216 may include a generator, an external power grid, a battery, or some other source of power. In certain embodiments, the power source 216 may be electrically coupled to the controller 202, the drive motor 122 for the cutter 106, the push block motor 128, and any other suitable electrical component that requires a power source. In some embodiments, the power source 216, similar to the motors 122, 128 may be disposed in a power room environment 236 separate from the cutting environment 238.

In the illustrated embodiment, the controller 202 is communicatively coupled to the drive motor 122. A first communication path 222 is configured to connect the drive motor 122 to the controller 202 such that the sensor 208 receives an input signal indicative of current operating conditions experienced by the drive motor 122. For example, the drive motor 122 may experience a condition where the cutter 106 requires an additional amount of power to cut through a particular cross section of a bone segment. In some embodiments, the controller 202 receives the signal from the sensor 208, accesses programmed control recipes stored in the memory 204 via the processor 206, and determines an appropriate control signal to output along a first control path 228 to the drive motor 122. The signal received from the sensor may use a detected voltage and/or current from the drive motor 122, and the controller 202 may determine a requisite power output that would be suitable. In other embodiments, the controller 202 may continuously receive input signals via the first communication path 222 and continuously output control signals along the first control path 228 to enable the drive motor 122 to cut bone fibers that are consistent in thickness, length, and quality.

In the illustrated embodiment, the controller 202 is communicatively coupled to the push block motor 128. A second communication path 224 is configured to connect the push block motor 128 to the controller 202 such that the sensor 208 receives an input signal indicative of current operating conditions experienced by the push block motor 128. For example, the push block motor 128 may experience a condition where the push block assembly 110 requires an additional amount of power to keep the bone segment in contact with the cutter 106. In some embodiments, the controller 202 receives the signal from the sensor 208, accesses programmed control recipes stored in the memory 204 via the processor 206, and determines an appropriate control signal to output along a second control path 226 to the push block motor 128. The signal received from the sensor may use a detected voltage and/or current from the push block motor 128, and the controller 202 may determine a requisite power output that would be suitable. In other embodiments, the controller 202 may continuously receive input signals via the second communication path 224 and continuously output control signals along the second control path 228 to enable the push block motor 128 to keep the bone segment in a position such that the cutter 106 may cut bone fibers that are consistent in thickness, length, and quality.

In the illustrated embodiment, a barrier 234, 146 is configured to divide the bone milling apparatus 100 into two or more environments. In a first environment, a cutting room environment 238 includes the cutter 106, housing 102, cover 104, push block assembly 110, feed chute 126 and fiber pan 124 as discussed previously in FIG. 1. The cutting room environment 238 is an enclosed work area, and it is envisioned and within the scope of the present techniques that the enclosed work area could be as large as a room, and the cutter 106 may extend into the cutting room environment 238 from one or more of the other environments. Additionally or alternatively, the bone segment 230 and cutter 106 are advantageously situated within the cutting room environment 238, isolated from other environments and potential contaminants, so as to reduce the opportunity for degradation or contamination of the bone segment 230 or completed bone fibers 232. In other embodiments, the cutting room environment 238 would meet the requirements for an ISO 5 cleanroom designation. In a second environment, a power room environment 236 includes the drive motor 122, the push block motor 128, the control system 200, the computing device 210 and the power source 216.

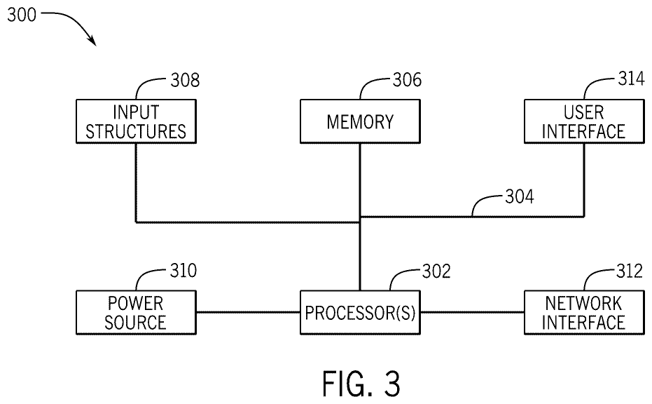

FIG. 3 illustrates a block diagram of example components of a computing device 300 that could be used as the computing device 210, the cloud/remote server 214, the controller 202, or some other device provided by the present techniques. As used herein, the computing device 300 may be implemented as one or more computing systems, including a laptop, notebook, desktop, tablet, HMI, or workstation computer, as well as server type devices (e.g., on-prem servers, remote servers, cloud servers) or portable, communication type devices, such as cellular telephones and/or suitable computing devices.

As illustrated, the computing device 300 may include various hardware components, such as one or more processors 302, one or more busses 304, memory 306, input structures 308, a power source 310, a network interface 312, a user interface 314, and/or other computer components useful in performing the functions described herein. The one or more processors 302 may include, in certain implementations, microprocessors configured to execute instructions stored in the memory 306 or other accessible locations. Alternatively, the one or more processors 302 may be implemented as application-specific integrated circuits (ASICs), field-programmable gate arrays (FPGAs), and/or devices designed to perform functions discussed herein in a dedicated manner. As will be appreciated, multiple processors 302 or processing components may be used to perform functions discussed herein in a distributed or parallel manner.

The memory 306 may encompass any tangible, non-transitory medium for storing data or executable routines. Although shown for convenience as a single block in FIG. 2, the memory 306 may encompass various discrete media in the same or different physical locations. The one or more processors 302 may access data in the memory 306 via one or more busses 304.

The input structures 308 may allow a user to input data and/or commands to the device 300 and may include mice, touchpads, touchscreens, keyboards, controllers, and so forth. The power source 310 can be any suitable source for providing power to the various components of the computing device 300, including line and battery power. In the depicted example, the device 300 includes a network interface 312. Such a network interface may allow communication with other devices on a network using one or more communication protocols. In the depicted example, the device 300 includes a user interface 314, such as a display that may display images or data provided by the one or more processors 302. The user interface 314 may include, for example, a monitor, a display, and so forth. As will be appreciated, in a real-world context a processor-based system, such as a computing device 300 of FIG. 3, may be employed to implement some or all of the present approach, such as performing the functions of the controller 202, the computing device 210, and or the cloud/remote server 214 shown in FIG. 2, as well as other memory-containing devices.

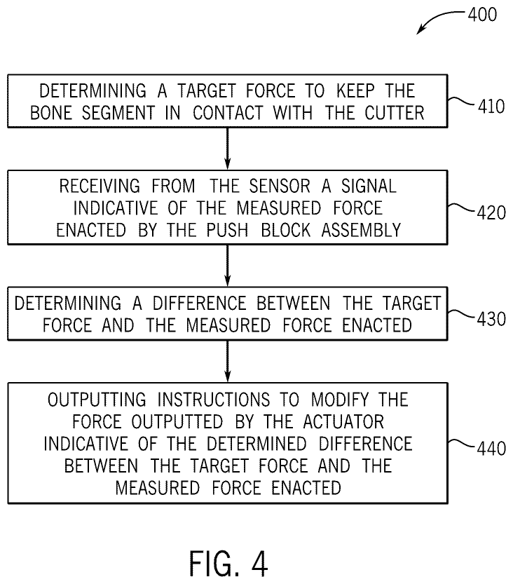

FIG. 4 depicts a flowchart of an embodiment of a method 400 for automatically controlling a bone milling apparatus during a milling operation of a bone sample. First in block 410 of the method 400, a bone milling apparatus includes a controller configured to determine a target force to keep a bone segment in contact with a cutter. In other embodiments, the controller may be configured to determine a cutting force that may be targeted to be outputted by a cutter in order to mill appropriately sized bone fibers. In certain embodiments, the controller may determine the target forces by accessing stored programming that may include “recipes” (i.e., preset or preconfigured operational setting and/or parameters) and/or predefined actions to take when the controller experiences a particular condition, as may be determined from a feedback or other sensor signal in some embodiments. In other embodiments, a computing device may input a desired length, thickness, or other controllable quality of a finished bone fiber, and the controller may receive the input and determine the target force output to maintain the bone segment in a position that enables the bone milling apparatus to output appropriately sized fibers. Additionally or alternatively, the recipes or predefined actions may include instructions for milling a bone segment with a specified length, thickness, and cross-sectional geometry, and based on historical data, determine a recipe for modifying the forces outputted by the push block motor and the cutting motor as the bone segment is milled.

At 420, the controller receives from a sensor a signal indicative of the force enacted by a push block assembly against the bone sample. The signal that the sensor receives may be in the form of a voltage, current, or otherwise appropriate means that may be used as a corollary to the force enacted to hold the bone sample against the cutter. In certain embodiments, the controller continuously receives signals indicative of the force enacted by the push block assembly. Due to the cross section of the bone sample varying during different stages of operation, the force enacted by the push block assembly may vary in a similar manner during the duration of the milling operation. At 430, the controller determines a difference between the target force to hold the bone sample against the cutter and the force currently being enacted. In some embodiments, the target force may be greater than the force enacted upon the push block assembly, the target force may be less than the force enacted upon the push block assembly, or the target force may be substantially equal to the force enacted upon the push block assembly. For example, while milling the bone segment, the cross-sectional area of the sample being milled may vary during the operation, and during portions of the duration where the cross-sectional area being milled is at a maximum, the power drawn from the push block motor and cutting motor to generate the target forces may be at a maximum. Additionally or alternatively, while the cross-sectional area of the sample being milled is at a minimum, the power drawn from the push block motor and cutting motor to generate the target forces may be at a minimum.

At 440, the controller may output instructions to modify the force outputted by the motor that is indicative of the determined difference between the target force and the measured force enacted. In certain embodiments, if the target force is greater than the measured force enacted upon the push block assembly, the controller may output a recipe including instructions that include increasing the power outputted by a push block motor. The increase in the power outputted may take the form of an increased torque, increase in speed or RPM of the motor, or otherwise appropriate means for increasing the power output. In other embodiments, if the target force is less than the measured force enacted upon the push block assembly, the controller may output a recipe including instructions that include decreasing the power outputted by the push block motor. The decrease in the power outputted may take the form of a decreased torque, decrease in speed or RPM of the motor, or otherwise appropriate means for decreasing the output. In a non-limiting embodiment, if the target force is substantially equal to the measured force enacted, and the controller may output a recipe including instructions to keep the power output of the motor at current levels.

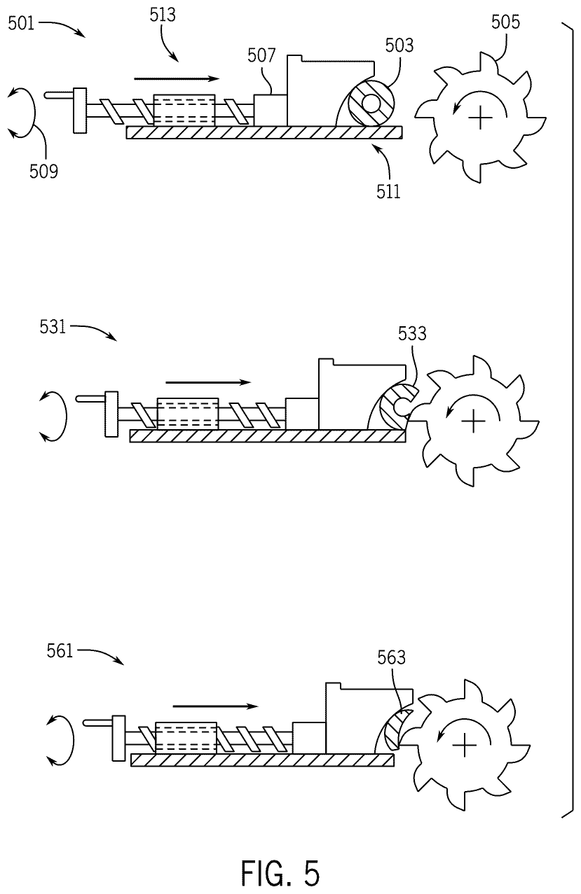

FIG. 5 is a front view of a simplified bone milling apparatus 100 in various stages of operation. In block 501, a full bone segment 503 is shown on top of the bottom plate 511 and is in a state prior to the milling process beginning. The full bone segment 503 is placed against the push block assembly 507 which is configured to translate horizontally along the bottom plate 511. In block 501, the push block assembly 507 is positioned away from the cutter 505, which is disposed to the right of the bottom plate 511. The push block assembly 507, as described previously, is configured to translate horizontally and apply an axial force 513 on the full bone segment 503, as the crank handle 509 is rotated. In certain embodiments, a drive motor may be coupled to the crank handle 509 or an otherwise appropriate member to electrically drive the rotation of the crank handle 509, thereby electrically applying the axial force 513.

In block 531, a partially cut bone segment 533 is shown on top of the bottom plate 511 and is in a state during the milling process. The partially cut bone segment 533 is kept in place against the cutter 505 by the push block assembly 507 as the crank handle 509 rotates. In certain embodiments, the cutter 505 is configured to rotate such that the partially cut bone segment 533 is able to produce bone fibers that have a consistent length, shape, thickness and quality. In block 561, a cut bone segment 563 is shown on top of the bottom plate 511 and is in a state nearing the end of the milling process. The cut bone segment 563 is kept in place against the cutter 505 by the push block assembly 507 as the crank handle 509 rotates. In a non-limiting embodiment, the cutter is configured to rotate such that the blade matches a profile of the push block of the push block assembly 507. In other embodiments, the push block profile may be configured to prevent the bone segment 503, 533, 563 from rotating while it is being cut, contributing to the consistency of the bone fibers produced from the bone milling apparatus.

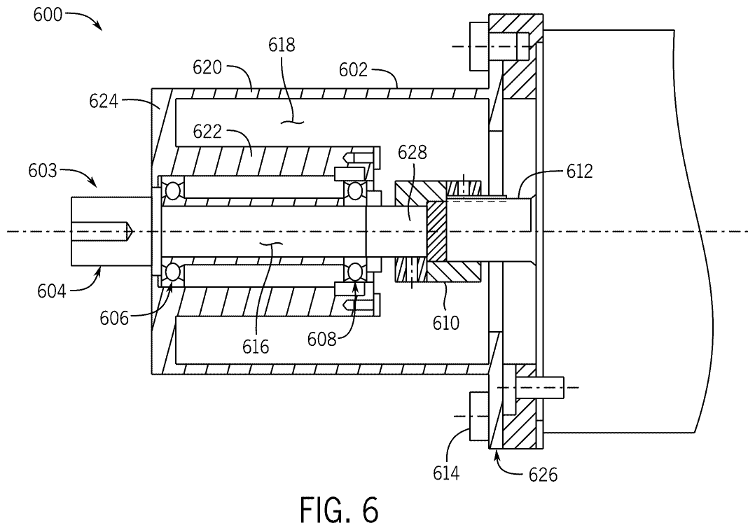

FIG. 6 is a schematic view of an embodiment of a bearing housing assembly 600 that is included as a part of the bone milling apparatus 100. The bearing housing assembly may include a support housing 602, a cutter shaft 603, 108, a first bearing 606, a second bearing 608, a coupling 610, and a plurality of fasteners 614. The support housing 602 is configured to provide rigidity and support for the cutter shaft 603, 108 and provide support for the first bearing 606 and second bearing 608. Additionally or alternatively, the support housing 602 is configured to provide cover for the cutter shaft 603, 108 such that contaminants (dust, grease, shavings, or fumes, etc.) do not come into contact with the cutter shaft as it rotates. In a non-limiting embodiment, the support housing 602 has an outer cylindrical shell 620 and an inner cylindrical annular portion 622 connected by a radial end plate 624. Further, the outer cylindrical shell 620 may additionally be fixed to a flange 626 on an end opposite from the radial end plate 624. In other embodiments, the support housing 602 and associated components 620, 622, 6241, 626 may be made from any suitable material, such as but not limited to cast iron, steel, metal, metal alloy, or any combination thereof. In a non-limiting embodiment, the radial end plate 624 may make up at least a portion of the barrier wall between the cutting room environment and the power room environment. Additionally or alternatively, the radial flange 626 may couple with a mounting plate of a motor, and the plurality of fasteners 614 may assemble through associated thru holes in the radial flange 626 and assemble into the mounting plate.

The inner cylindrical annular portion 622 may include a plurality of bearing housing locations configured to accept the first bearing 606 and the second bearing 608. In certain embodiments, the first bearing 606 and the second bearing 608 may be a cylindrical roller bearing, a single row tapered roller bearing, a spherical bearing, or any otherwise suitable bearing design. The first bearing 606 and second bearing 608 may be of similar configurations and diameters, or may have different configurations and different diameters. In other embodiments, the first bearing 606 and second bearing may both be cylindrical roller bearings. In yet other embodiments, the first bearing 606 may be a single-row tapered roller bearing and the second bearing 608 may be a spherical roller bearing. By utilizing the support housing 602 with the inner cylindrical annular portion 622, present embodiments enable a larger distance between the first bearing 606 and second bearing 608, and eliminate the requirement for additional wear bushings, and corresponding bushing housings that require additional maintenance.

In some embodiments, the cutter shaft 603, 108 includes a plurality of portions, with each portion configured to provide different functionalities. Taken together, the cutter shaft 603, 108 may be configured to transfer a rotational output from a motor shaft 612 to and output a cutting force via the cutter 106, as shown previously in FIG. 1. In certain embodiments, the cutter shaft 603, 108 includes a coupling portion 628, a middle portion 616, and a cutter head portion 604. In other embodiments, the cutter shaft 603, 108 may include additional portions configured to perform other various suitable operations related to bone milling. The coupling portion 628 may be configured to couple to the motor shaft 612 and may have a specified diameter. In the illustrated embodiment, the coupling portion 628 has a substantially similar diameter as the motor shaft 612, and this similarity enables the coupling portion 628 and motor shaft 612 to be attached via a coupling 610. The diameter of the coupling portion 628 may be different than the diameters of the middle portion 616 and the cutter head portion 604. For example, in one embodiment, the coupling portion 628 may have a smaller diameter than the middle portion 616 and the cutter head portion 604. As a result of the coupling portion 628 having a smaller diameter than the middle portion 616, this enables the first bearing 606 and second bearing 608 to assemble onto the middle portion 616 by sliding over the coupling portion 628.

As stated above, the middle portion 616 of the cutter shaft 603 may be configured to interface with the first bearing 606 and second bearing 608. In the illustrated embodiment, the middle portion 616 has a substantially similar diameter along the length of the middle portion. In certain embodiments, the middle portion 616 may have a smaller diameter than the head portion 604 of the cutter shaft 603. The cutter head portion 604 is configured to accept a cutter. In other embodiments, the cutter head portion 604 may be configured to have a diameter specified to be larger than an interfacing inner diameter of the cutter, thereby introducing an interference fit between the cutter head portion 604 and the cutter. In a non-limiting embodiment, the diameter of the cutter head portion 604 may be specified to be smaller than the interfacing inner diameter of the cutter, and provide for an otherwise appropriate means for attaching the cutter to the cutter shaft 603. In a non-limiting embodiment, the cutter shaft 603 may be made of any suitable material, including but not limited to metal, steel, metal alloy, or any combination thereof.

The subject matter described in detail above may be defined by one or more clauses, as set forth below.
Clause 1: A bone milling apparatus comprises a support housing, wherein the housing comprises a base portion with a top surface, a top portion vertically spaced from the base portion, and support braces configured to couple the top portion with the base portion, a cutter configured to rotate about a rotational axis, a push block assembly disposed on top of the base portion, wherein the push block assembly is configured to translate along the top surface of the base portion, thereby enacting a force against a bone segment during use to keep the bone segment in contact with the cutter, a push block motor communicatively coupled to the push block assembly, and a drive motor communicatively coupled to the cutter, wherein the drive motor is configured to rotate the cutter about the rotational axis.
Clause 2: The bone milling apparatus of clause 1, wherein the support housing, cutter, and push block assembly are disposed in a cutting room environment on a first side of a barrier wall, and the push block motor and the drive motor are disposed in a power room environment on a second side of the barrier wall.
Clause 3: The bone milling apparatus of clause 1 or 2, wherein the cutting room environment comprises a cleanroom environment.
Clause 4: The bone milling apparatus of clause 1, 2, or 3, wherein the push block assembly comprises a push block with an interface surface, wherein the interface surface comprises an engaging feature that assists in preventing rotation of the bone segment during use and in maintaining the bone segment against the cutter, wherein the engaging feature comprises at least one of serrations, spikes, nodules, a textured surface, knurling, or any combination thereof.
Clause 5: The bone milling apparatus of clause 1, 2, 3, or 4, wherein the cutter comprises a plurality of teeth configured in a helical pattern along a rotational axis of the cutter.
Clause 6: The bone milling apparatus of clause 1, 2, 3, 4, or 5, wherein the cutter comprises eight teeth, wherein each of the eight teeth is configured to have a helix angle of 30 degrees with respect to the rotational axis.
Clause 7: The bone milling apparatus of clause 1, 2, 3, 4, 5, or 6, further including a bearing housing assembly that comprises a bearing support housing, a cutter shaft configured to rotate about the rotational axis, a first bearing configured to enable rotation of the cutter shaft, wherein the first bearing is disposed in the bearing support housing, a second bearing additionally configured to enable rotation of the cutter shaft, wherein the second bearing is also disposed in the support housing, wherein the second bearing is horizontally spaced along the rotational axis from the first bearing; and a coupling, wherein the coupling is configured to mechanically couple the cutter shaft to a motor shaft.
Clause 8: The bone milling apparatus of clause 1, 2, 3, 4, 5, 6, or 7, wherein the first bearing and the second bearing are cylindrical roller bearings.
Clause 9: The bone milling apparatus of clause 1, 2, 3, 4, 5, 6, 7, or 8, wherein the cutter is configured to removably couple to the cutter shaft.
Clause 10: The bone milling apparatus of clause 1, 2, 3, 4, 5, 6, 7, 8, or 9, wherein the bearing support housing comprises an outer cylindrical shell with a first end and a second end, an inner cylindrical annular portion, a radial end plate fixed to the first end of the outer cylindrical shell and the inner cylindrical annular portion, and a flange fixed to the second end of the outer cylindrical shell.
Clause 11: The bone milling apparatus of clause 1, 2, 3, 4, 5, 6, 7, 8, 9, or 10, wherein the flange is configured to couple to a mounting plate of the drive motor.
Clause 12: The bone milling apparatus of clause 1, 2, 3, 4, 5, 6, 7, 8, 9, 10, or 11, wherein the outer cylindrical shell, inner cylindrical annular portion, the radial end plate, and the flange are made from cast iron, steel, metal, metal alloy, or any combination thereof.
Clause 13: A method, including placing a bone segment into an input chute of a bone milling apparatus, wherein the input chute is configured to direct the bone segment to be positioned against a push block of a push block assembly, powering on a drive motor that is coupled to a cutter shaft, wherein the drive motor is configured to output a rotational force to the cutter shaft, outputting the rotational force to the cutter shaft, whereby the cutter shaft begins to rotate at a specified rotational speed and a specified torque, powering on a push block motor that is coupled to the push block assembly, wherein the push block motor is configured to directly or indirectly output an axial force to the push block assembly, outputting the axial force to the push block assembly, wherein the axial force keeps the bone segment in contact with a cutter disposed on the cutter shaft, and milling the bone segment and generating bone fibers as a result of the push block assembly applying the axial force to the bone segment against the cutter.
Clause 14: The method of the clause 13, wherein powering on the drive motor that is coupled to a cutter shaft comprises electrically coupling the drive motor to a power source.
Clause 15: The method of clause 13 or 14, wherein powering on the push block motor comprises electrically coupling push block motor to a power source.
Clause 16: The method of clause 13, 14, or 15, wherein the rotational force output from the drive motor and the axial force output from the push block motor are adjusted to generate bone fibers with a specified length, thickness, cross-sectional geometry or combination thereof.
Clause 17: The method of clause 13, 14, 15, or 16, wherein the push block assembly comprises a drive screw, wherein the drive screw is configured to convert rotational motion into a translation motion.
Clause 18: The method of clause 13, 14, 15, 16, or 17, wherein the axial force is accomplished via the drive screw converting a rotational output from the push block motor into the axial force.
Clause 19: The method of clause 13, 14, 15, 16, 17, or 18, wherein the axial force accomplished via the drive screw is substantially suitable to counteract the rotational force propagated from the cutter disposed on the cutter shaft.
Clause 20: The method of clause 13, 14, 15, 16, 17, 18, or 19, including automatically collecting the bone fibers that are milled from the bone segment in a fiber pan.

While only certain features of the invention have been illustrated and described herein, many modifications and changes will occur to those skilled in the art. It is, therefore, to be understood that the appended claims are intended to cover all such modifications and changes as fall within the true spirit of the invention.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for (perform)ing (a function) . . . ” or “step for (perform)ing (a function) . . . ”, it is intended that such elements are to be interpreted under 35 U.S.C. 112 (f). However, for any claims containing elements designated in any other manner, it is intended that such elements are not to be interpreted under 35 U.S.C. 112 (f).

1. A bone milling apparatus, comprising:
a support housing, wherein the housing comprises a base portion with a top surface, a top portion vertically spaced from the base portion, and support braces configured to couple the top portion with the base portion;
a cutter configured to rotate about a rotational axis;
a push block assembly disposed on top of the base portion, wherein the push block assembly is configured to translate along the top surface of the base portion, thereby enacting a force against a bone segment during use to keep the bone segment in contact with the cutter;
a push block motor communicatively coupled to the push block assembly; and
a drive motor communicatively coupled to the cutter, wherein the drive motor is configured to rotate the cutter about the rotational axis.

2. The bone milling apparatus of claim 1, wherein the support housing, cutter, and push block assembly are disposed in a cutting room environment on a first side of a barrier wall, and the push block motor and the drive motor are disposed in a power room environment on a second side of the barrier wall.

3. The bone milling apparatus of claim 2, wherein the cutting room environment comprises a cleanroom environment.

4. The bone milling apparatus of claim 1, wherein the push block assembly comprises a push block with an interface surface, wherein the interface surface comprises an engaging feature that assists in preventing rotation of the bone segment during use and in maintaining the bone segment against the cutter, wherein the engaging feature comprises at least one of:
serrations;
spikes;
nodules;
a textured surface;
knurling; or
any combination thereof.

5. The bone milling apparatus of claim 1, wherein the cutter comprises a plurality of teeth configured in a helical pattern along a rotational axis of the cutter.

6. The bone milling apparatus of claim 5, wherein the cutter comprises eight teeth, wherein each of the eight teeth is configured to have a helix angle of 30 degrees with respect to the rotational axis.

7. The bone milling apparatus of claim 1, comprising a bearing housing assembly further comprising:
a bearing support housing;
a cutter shaft configured to rotate about the rotational axis;
a first bearing configured to enable rotation of the cutter shaft, wherein the first bearing is disposed in the bearing support housing;
a second bearing additionally configured to enable rotation of the cutter shaft, wherein the second bearing is also disposed in the support housing, wherein the second bearing is horizontally spaced along the rotational axis from the first bearing; and
a coupling, wherein the coupling is configured to mechanically couple the cutter shaft to a motor shaft.

8. The bone milling apparatus of claim 7, wherein the first bearing and the second bearing are cylindrical roller bearings.

9. The bone milling apparatus of claim 7, wherein the cutter is configured to removably couple to the cutter shaft.

10. The bone milling apparatus of claim 7, wherein the bearing support housing comprises:
an outer cylindrical shell with a first end and a second end;
an inner cylindrical annular portion;
a radial end plate fixed to the first end of the outer cylindrical shell and the inner cylindrical annular portion; and
a flange fixed to the second end of the outer cylindrical shell.

11. The bone milling apparatus of claim 10, wherein the flange is configured to couple to a mounting plate of the drive motor.

12. The bone milling apparatus of claim 10, wherein the outer cylindrical shell, inner cylindrical annular portion, the radial end plate, and the flange are made from cast iron, steel, metal, metal alloy, or any combination thereof.

13. A method, comprising:
placing a bone segment into an input chute of a bone milling apparatus, wherein the input chute is configured to direct the bone segment to be positioned against a push block of a push block assembly;
powering on a drive motor that is coupled to a cutter shaft, wherein the drive motor is configured to output a rotational force to the cutter shaft;
outputting the rotational force to the cutter shaft, whereby the cutter shaft begins to rotate at a specified rotational speed and a specified torque;
powering on a push block motor that is coupled to the push block assembly, wherein the push block motor is configured to directly or indirectly output an axial force to the push block assembly;
outputting the axial force to the push block assembly, wherein the axial force keeps the bone segment in contact with a cutter disposed on the cutter shaft; and
milling the bone segment and generating bone fibers as a result of the push block assembly applying the axial force to the bone segment against the cutter.

14. The method of claim 13, wherein powering on the drive motor that is coupled to a cutter shaft comprises electrically coupling the drive motor to a power source.

15. The method of claim 13, wherein powering on the push block motor comprises electrically coupling push block motor to a power source.

16. The method of claim 13, wherein the rotational force output from the drive motor and the axial force output from the push block motor are adjusted to generate bone fibers with a specified length, thickness, cross-sectional geometry or combination thereof.

17. The method of claim 13, wherein the push block assembly comprises a drive screw, wherein the drive screw is configured to convert rotational motion into a translation motion.

18. The method of claim 17, wherein the axial force is accomplished via the drive screw converting a rotational output from the push block motor into the axial force.

19. The method of claim 18, wherein the axial force accomplished via the drive screw is substantially suitable to counteract the rotational force propagated from the cutter disposed on the cutter shaft.

20. The method of claim 13, comprising automatically collecting the bone fibers that are milled from the bone segment in a fiber pan.
